(19) United States
(12) Patent Application Publication
(10) Pub. No.: US 2006/0149109 A1
(43) Pub. Date: Jul. 6, 2006

(54) Converting methanol and ethanol to light olefins

(76) Inventors: Philip Andrew Ruziska, Kingwood, TX (US); Christopher D.W. Jenkins, Houston, TX (US); James R. Lattner, Seabrook, TX (US); Michael Peter Nicoletti, Houston, TX (US); Michael J. Veraa, Houston, TX (US); Cor F. van Egmond, Pasadena, TX (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 11/075,286

(22) Filed: Mar. 8, 2005

Related U.S. Application Data
(60) Provisional application No. 60/640,866, filed on Dec. 30, 2004.

(51) Int. Cl. C07C 1/00

(52) U.S. Cl. 585/639

(57) ABSTRACT

The present invention provides processes for producing light olefins from a feedstock comprising methanol and ethanol. The ethanol is converted to ethylene and water over a dehydration catalyst, while the methanol is converted to light olefins and water over a molecular sieve catalyst. These conversion steps may occur in two separate reactors operating in series or in parallel, or in a single reactor containing a mixture of dehydration catalyst and molecular sieve catalyst.

### CROSS REFERENCE TO RELATED APPLICATIONS

This application claims the benefit of Provisional Application No. 60/640,866 filed Dec. 30, 2004, the disclosure of which is fully incorporated herein by reference.

### FIELD OF THE INVENTION

The present invention relates to processes for forming light olefins. More particularly, the present invention relates to processes for converting a mixture of methanol and ethanol to light olefins.

### BACKGROUND OF THE INVENTION

Light olefins, defined herein as ethylene and propylene, are important commodity petrochemicals useful in a variety of processes for making plastics and other chemical compounds. Ethylene is used to make various polyethylene plastics, and in making other chemicals such as vinyl chloride, ethylene oxide, ethyl benzene and alcohol. Propylene is used to make various polypropylene plastics, and in making other chemicals such as acrylonitrile and propylene oxide.

The petrochemical industry has known for some time that oxygenates, especially alcohols, are convertible into light olefins. The preferred conversion process is generally referred to as an oxygenate to olefin (OTO) reaction process. Specifically, in an OTO reaction process, an oxygenate contacts a molecular sieve catalyst composition under conditions effective to convert at least a portion of the oxygenate to light olefins. When methanol is the oxygenate, the process is generally referred to as a methanol to olefin (MTO) reaction process. Methanol is a particularly preferred oxygenate for the synthesis of ethylene and/or propylene.

Depending on the respective commercial markets for ethylene and propylene, it may be desirable to vary the weight ratio of ethylene to propylene formed in an OTO reaction system. It has recently been discovered, however, that although percent conversion may vary with a change in reaction conditions, e.g., temperature or pressure, the selectivity of a methanol-containing feedstock for ethylene and propylene in an OTO reaction system is relatively insensitive to changes in reaction conditions. Thus, the need exists in the art for a process for varying the ratio of ethylene to propylene formed in an OTO reaction system.

U.S. patent application Ser. No. 10/716,894, filed on Nov. 19, 2003, the entirety of which is incorporated herein by reference, is directed to processes for producing light olefins from methanol and ethanol, optionally in a mixed alcohol stream. The invention includes directing a first syngas stream to a methanol synthesis zone to form methanol and directing a second syngas stream and methanol to a homologation zone to form ethanol. The methanol and ethanol are directed to an oxygenate to olefin reaction system for conversion thereof to ethylene and propylene.

U.S. patent application Ser. No. 10/717,006, filed on Nov. 19, 2003, the entirety of which is incorporated herein by reference, is directed to processes for producing methanol and ethanol in a mixed alcohol stream. Syngas is directed to a synthesis zone wherein the syngas contacts a methanol synthesis catalyst and an ethanol synthesis catalyst (either a homologation catalyst or a fuel alcohol synthesis catalyst) under conditions effective to form methanol and ethanol. The methanol and ethanol, in a desired ratio, are directed to an oxygenate to olefin reaction system for conversion thereof to ethylene and propylene in a desired ratio. The invention also relates to processes for varying the weight ratio of ethylene to propylene formed in an oxygenate to olefin reaction system.

U.S. patent application Ser. No. 10/716,685, filed on Nov. 19, 2003, the entirety of which is incorporated herein by reference, is directed to processes for producing C1 to C4 alcohols in a mixed alcohol stream and optionally converting the alcohols to light olefins. A first portion of a syngas stream is directed to a methanol synthesis zone wherein methanol is synthesized. A second portion of the syngas stream is directed to a fuel alcohol synthesis zone wherein fuel alcohol is synthesized. The methanol and at least a portion of the fuel alcohol are directed to an oxygenate to olefin reaction system for conversion thereof to ethylene and propylene.

PCT Application No. PCT/US2004/035474, filed on Oct. 25, 2004, the entirety of which is incorporated herein by reference, is directed to controlling the ratio of ethylene to propylene produced in an oxygenate to olefin conversion process. The focus of the '474 application is on synthesizing an alcohol-containing feedstock comprising a mixture of methanol and ethanol and directing the alcohol-containing feedstock to an OTO reaction system for conversion thereof to ethylene and propylene in a desired ratio.

The conversion of methanol to light olefins (MTO) typically requires harsher reaction conditions, e.g., temperature and/or pressure, than are required for the dehydration of ethanol to light olefins. These harsher conditions are believed to cause the ethanol in the alcohol-containing feedstock to break down and form undesirable side reaction byproducts. For example, it has now been discovered that the conversion of ethanol to light olefins at MTO reaction conditions produces a considerable amount of acetaldehyde byproduct, which may be difficult to remove from the resulting light olefin-containing effluent. Thus, the need exists for converting a mixed alcohol-containing feedstock to light olefins while minimizing the formation of undesirable side-reaction byproducts.

### SUMMARY OF THE INVENTION

The present invention is directed to processes for converting a mixed alcohol-containing feedstock to light olefins while minimizing the formation of undesirable side reaction byproducts such as acetaldehyde.

In one embodiment, for example, the invention is to a process for producing light olefins, the process comprising the steps of: (a) providing a feedstock comprising methanol and ethanol; (b) dehydrating at least a portion of the ethanol in a first reactor to form a first effluent comprising ethylene, methanol, water and less than about 2 weight percent acetaldehyde, based on the total weight of the first effluent; and (c) contacting the methanol in the first effluent with a molecular sieve catalyst composition in a second reactor under conditions effective to convert the methanol to additional light olefins. Optionally, the process further comprises the step of: (d) removing a weight majority of the water from the first effluent between steps (b) and (c).

Optionally, the molecular sieve catalyst composition comprises a molecular sieve selected from the group consisting of: SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, AEI/CHA intergrowths, metal containing forms thereof, intergrown forms thereof, and mixtures thereof.

Optionally, the cumulative amount of ethylene and propylene formed in steps (b) and (c) has a weight ratio of ethylene to propylene of greater than about 0.7, greater than about 1.0, or greater than about 1.2 based on the total amount of ethylene and propylene formed in steps (b) and (c).

Optionally, the methanol to ethanol weight ratio in the feedstock is from about 1 to about 100, or from about 3 to about 20.

Optionally, step (b) comprises contacting the ethanol with a dehydration catalyst under conditions effective to convert the ethanol to the ethylene and water, wherein the dehydration catalyst is selected from the group consisting of: silica-alumina, activated alumina, phosphoric acid, and activated clay.

Optionally, the first effluent comprises less than about 2, less than about 1, less than about 0.2, less than about 0.1, or less than about 0.05 weight percent acetaldehyde, based on the total weight of the first effluent. Additionally or alternatively, the first effluent optionally comprises at least about 5 or at least about 25 weight percent methanol, based on the total weight of the first effluent. Additionally or alternatively, the first effluent optionally comprises at least about 5, or at least about 10 weight percent ethylene, based on the total weight of the first effluent.

Optionally, at least a portion of the methanol from the feedstock is dehydrated to dimethyl ether in the first reactor, and wherein the first effluent further comprises the dimethyl ether. In this aspect of the present invention, the first effluent optionally comprises at least about 5 weight percent or at least about 25 weight percent dimethyl ether, based on the total weight of the first effluent. Optionally, the process further comprises the step of: contacting at least a portion of the dimethyl ether with the molecular sieve catalyst composition in the second reactor under conditions effective to convert the dimethyl ether to ethylene.

Optionally, a weight majority of the methanol from the first feedstock passes through the first reactor and into the first effluent.

Optionally, the first reactor comprises an alcohol dehydration reactive distillation column. In this aspect of the invention, a weight majority of the water formed in step (b) optionally is separated in the distillation column from a weight majority of the methanol and ethylene, collectively, formed in step (b).

In another embodiment, the invention is to a process for producing light olefins, the process comprising the steps of: (a) providing a feedstock comprising methanol and ethanol; (b) separating the feedstock into a methanol-containing stream and an ethanol-containing stream, wherein the methanol-containing stream comprises a weight majority of the methanol from the feedstock, and the ethanol-containing stream comprises a weight majority of the ethanol from the feedstock; (c) contacting the ethanol in the ethanol-containing stream with a dehydration catalyst in a first reactor under conditions effective to convert the ethanol to water and light olefins, wherein the light olefins are yielded from the first reactor in a first effluent; (d) contacting the methanol in the methanol-containing stream with a molecular sieve catalyst composition in a second reactor under conditions effective to convert the methanol to light olefins and water, which are yielded from the second reactor in a second effluent; and (e) combining at least a portion of the first effluent with at least a portion of the second effluent to form a combined product stream.

Optionally, the molecular sieve catalyst composition comprises a molecular sieve selected from the group consisting of: SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, AEI/CHA intergrowths, metal containing forms thereof, intergrown forms thereof, and mixtures thereof.

Optionally, the cumulative amount of ethylene and propylene formed in steps (c) and (d) has a weight ratio of ethylene to propylene of greater than about 0.7, greater than about 1.0, or greater than about 1.2, based on the total amount of ethylene and propylene formed in steps (c) and (d).

Optionally, the methanol to ethanol weight ratio in the feedstock is from about 1 to about 100 or from about 3 to about 20.

Optionally, the dehydration catalyst is selected from the group consisting of: silica-alumina, activated alumina, phosphoric acid, and activated clay.

Optionally, the first effluent comprises less than about 2 weight percent, less than about 1 weight percent or less than about 0.2 weight percent acetaldehyde, based on the total weight of the first effluent.

Optionally, the first reactor comprises an alcohol dehydration reactive distillation column. In this aspect of the invention, the alcohol dehydration reactive distillation column optionally separates a weight majority of the light olefins formed in step (c) from a weight majority of the water formed in step (c), wherein the first effluent comprises the weight majority of the light olefins.

Optionally, the first reactor comprises a fixed bed dehydration reactor.

Optionally, the first effluent further comprises the water formed in step (c).

Optionally, the feedstock further comprises one or more C3+ alcohols, a weight majority of which are separated in step (b) into the ethanol-containing stream, and which C3+ alcohols are also dehydrated to light olefins and water in the first reactor. In this aspect of the invention, the feedstock optionally comprises more than 1 weight percent C3+ alcohols, based on the weight of the feedstock.

Optionally, the feedstock further comprises greater than about 1 weight percent or greater than about 10 weight percent water, based on the total weight of the feedstock.

In another embodiment, the invention is to a process for producing light olefins, the process comprising the steps of: (a) providing a feedstock comprising methanol and ethanol; and (b) fluidizing a population of catalyst particles in a fluidized reactor with the feedstock under conditions effective to convert the methanol and the ethanol to light olefins and water, wherein the population of catalyst particles comprises ETE catalyst particles and molecular sieve catalyst particles.

Optionally, the population of catalyst particles comprises from about 2 to about 22 weight percent ETE catalyst particles, more preferably from about 8 to about 16 weight percent ETE catalyst particles, based on the total weight of the population of catalyst particles.

Optionally, the ETE catalyst particles are selected from the group consisting of: silica-alumina catalyst particles, activated alumina catalyst particles, solid phosphoric acid, and activated clay catalyst particles. In this aspect of the invention, the molecular sieve catalyst particles preferably comprise a molecular sieve selected from the group consisting of: SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, AEI/CHA intergrowths, metal containing forms thereof, intergrown forms thereof, and mixtures thereof.

Optionally, the light olefins comprise ethylene and propylene, and the weight ratio of ethylene to propylene formed in step (b) is greater than about 0.7, preferably greater than about 1.0, and most preferably greater than about 1.2.

Optionally, the methanol to ethanol weight ratio in the feedstock is from about 1 to about 100, preferably from about 3 to about 20.

Optionally, the light olefins and water formed in step (b) are yielded from the fluidized reactor in an effluent stream comprising less than 2 weight percent, preferably less than about 1 weight percent, and more preferably less than 0.2 weight percent acetaldehyde, based on the total weight of the effluent stream.

Optionally, the feedstock further comprises greater than about 1 weight percent water, optionally greater than about 10 weight percent water, based on the total weight of the feedstock.

### BRIEF DESCRIPTION OF THE DRAWINGS

This invention will be better understood by reference to the detailed description of the invention when taken together with the attached drawings, wherein:

FIG. 1 is a flow diagram illustrating an oxygenate to olefins reaction system;

FIG. 2 is a flow diagram illustrating an ethanol to ethylene reaction system;

FIG. 3 is a flow diagram illustrating one embodiment of the present invention;

FIG. 4 is a flow diagram illustrating another embodiment of the present invention; and

FIG. 5 is a flow diagram illustrating another embodiment of the present invention.

### DETAILED DESCRIPTION OF THE INVENTION

A. Introduction

The present invention, in one embodiment, provides processes for producing light olefins from a feedstock comprising methanol and ethanol. In one embodiment, at least a portion of the ethanol is dehydrated in a first reactor to form a first effluent comprising ethylene, methanol, water and less than about 2 weight percent acetaldehyde, based on the total weight of the first effluent. The methanol in the first effluent contacts a molecular sieve catalyst composition in a second reactor under conditions effective to convert the methanol to additional light olefins.

In another embodiment, the feedstock is separated into a methanol containing stream and an ethanol containing stream. These streams are then converted to light olefins in a methanol to olefins (MTO) reactor and an ethanol to ethylene (ETE) reactor, respectively, which operate in parallel. The subsequently formed effluent streams are then optionally combined and directed to a single separation system.

In another embodiment, the feedstock is directed to a single reactor, which implements a population of catalyst particles comprising MTO catalyst particles and ETE catalyst particles. The methanol and ethanol contact these catalyst particles in the reactor under conditions effective to convert the methanol and ethanol to light olefins.

B. Methanol to Olefins Reaction Processes

As indicated above, one aspect of the invention is directed to converting methanol to light olefins, preferably a combination of ethylene and propylene. The MTO reaction process will now be described in greater detail.

In a MTO reaction system, an MTO catalyst composition, preferably a molecular sieve catalyst composition, is used to convert a methanol-containing feedstock to light olefins. As used herein, “reaction system” means a system comprising a reactor, optionally a catalyst cooler, optionally a catalyst regenerator, and optionally a catalyst stripper. The reactor comprises a reaction unit, which defines a reaction zone, and optionally a disengaging unit, which defines a disengaging zone. As used herein, the terms “catalyst particle” and “catalyst composition” are synonymous and interchangeably used.

Ideally, the molecular sieve catalyst composition comprises an alumina or a silica-alumina catalyst composition, optionally an amorphous alumina or a silica-alumina catalyst composition that does not act as a molecular sieve. Silicoaluminophosphate (SAPO) molecular sieve catalysts are particularly desirable in such conversion processes, because they are highly selective in the formation of ethylene and propylene. A non-limiting list of preferable SAPO molecular sieve catalyst compositions includes SAPO-17, SAPO-18, SAPO-34, SAPO-35, SAPO-44, the substituted forms thereof, and mixtures thereof. The molecular sieve catalyst composition fluidized according to the present invention optionally comprises a molecular sieve selected from the group consisting of: SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, AEI/CHA intergrowths, metal containing forms thereof, intergrown forms thereof, and mixtures thereof. Additionally or alternatively, the molecular sieve comprises an aluminophosphate (ALPO) molecular sieve. Preferred ALPO molecular sieves include ALPO-5, ALPO-11, ALPO-18, ALPO-31, ALPO-34, ALPO-36, ALPO-37, ALPO-46, AEI/CHA intergrowths, mixtures thereof, and metal containing forms thereof. Ideally, the catalyst to be fluidized according to the present invention is selected from the group consisting of: SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, a zeolitic molecular sieve, ZSM-34, ZSM-5, metal containing forms thereof, intergrown forms thereof, AEI/CHA intergrowths, and mixtures thereof.

In a preferred embodiment, the MTO catalyst composition comprises a molecular sieve having an average pore size of less than about 6 Å (0.6 nm), more preferably less than about 5 Å (0.5 nm). Preferably, the molecular sieve has an 8 or 10-member ring structure, preferably an 8-member ring structure.

The oxygenate-containing feedstock that is directed to an MTO reaction system optionally contains one or more aliphatic-containing compounds such as alcohols, amines, carbonyl compounds for example aldehydes, ketones and carboxylic acids, ethers, halides, mercaptans, sulfides, and the like, and mixtures thereof. The aliphatic moiety of the aliphatic-containing compounds typically contains from 1 to about 50 carbon atoms, preferably from 1 to 20 carbon atoms, more preferably from 1 to 10 carbon atoms, and more preferably from 1 to 4 carbon atoms, and most preferably methanol.

Non-limiting examples of aliphatic-containing compounds include: alcohols such as methanol and ethanol, alkyl-mercaptans such as methyl mercaptan and ethyl mercaptan, alkyl-sulfides such as methyl sulfide, alkyl-amines such as methyl amine, alkyl-ethers such as DME, diethyl ether and methylethyl ether, alkylhalides such as methyl chloride and ethyl chloride, alkyl ketones such as dimethyl ketone, alkyl-aldehydes such as formaldehyde and acetaldehyde, and various acids such as acetic acid.

In a preferred embodiment of the process of the invention, the feedstock contains one or more organic compounds containing at least one oxygen atom. In the most preferred embodiment of the process of invention, the oxygenate in the feedstock comprises one or more alcohols, preferably aliphatic alcohols where the aliphatic moiety of the alcohol(s) has from 1 to 20 carbon atoms, preferably from 1 to 10 carbon atoms, and most preferably from 1 to 4 carbon atoms. The alcohols useful as feedstock in the process of the invention include lower straight and branched chain aliphatic alcohols and their unsaturated counterparts. Non-limiting examples of oxygenates include methanol, ethanol, n-propanol, isopropanol, methyl ethyl ether, DME, diethyl ether, di-isopropyl ether, formaldehyde, dimethyl carbonate, dimethyl ketone, acetic acid, and mixtures thereof. In the most preferred embodiment, the feedstock comprises one or more of methanol, ethanol, DME, diethyl ether or a combination thereof.

The various feedstocks discussed above are converted primarily into one or more olefins. The olefins or olefin monomers produced from the feedstock typically have from 2 to 30 carbon atoms, preferably 2 to 8 carbon atoms, more preferably 2 to 6 carbon atoms, still more preferably 2 to 4 carbons atoms, and most preferably ethylene and/or propylene.

Non-limiting examples of olefin monomer(s) include ethylene, propylene, butene-1, pentene-1,4-methyl-pentene-1, hexene-1, octene-1 and decene-1, preferably ethylene, propylene, butene-1, pentene-1,4-methyl-pentene-1, hexene-1, octene-1 and isomers thereof. Other olefin monomers include unsaturated monomers, diolefins having 4 to 18 carbon atoms, conjugated or nonconjugated dienes, polyenes, vinyl monomers and cyclic olefins.

In a preferred embodiment, the feedstock, which ideally comprises methanol, is converted in the presence of a molecular sieve catalyst composition into olefin(s) having 2 to 6 carbons atoms, preferably 2 to 4 carbon atoms. Most preferably, the olefin(s), alone or combination, are converted from a feedstock containing an oxygenate, preferably an alcohol, most preferably methanol, to the preferred olefin(s) ethylene and/or propylene.

The most preferred process is generally referred to as an oxygenate-to-olefins (OTO) reaction process. In an OTO process, typically an oxygenated feedstock, most preferably a methanol- and ethanol-containing feedstock, is converted in the presence of a molecular sieve catalyst composition into one or more olefins, preferably and predominantly, ethylene and/or propylene, referred to herein as light olefins.

The feedstock, in one embodiment, contains one or more diluents, typically used to reduce the concentration of the feedstock. The diluents are generally non-reactive to the feedstock or molecular sieve catalyst composition. Non-limiting examples of diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred. In other embodiments, the feedstock does not contain any diluent.

The diluent may be used either in a liquid or a vapor form, or a combination thereof. The diluent is either added directly to a feedstock entering into a reactor or added directly into a reactor, or added with a molecular sieve catalyst composition. In one embodiment, the amount of diluent in the feedstock is in the range of from about 1 to about 99 mole percent based on the total number of moles of the feedstock and diluent, preferably from about 1 to 80 mole percent, more preferably from about 5 to about 50, most preferably from about 5 to about 25. In one embodiment, other hydrocarbons are added to a feedstock either directly or indirectly, and include olefin(s), paraffin(s), aromatic(s) (see for example U.S. Pat. No. 4,677,242, addition of aromatics) or mixtures thereof, preferably propylene, butylene, pentylene, and other hydrocarbons having 4 or more carbon atoms, or mixtures thereof.

The process for converting a feedstock, especially a feedstock containing one or more oxygenates, in the presence of a molecular sieve catalyst composition of the invention, is carried out in a reaction process in a reactor, where the process is a fixed bed process, a fluidized bed process (includes a turbulent bed process), preferably a continuous fluidized bed process, and most preferably a continuous high velocity fluidized bed process.

The reaction processes can take place in a variety of catalytic reactors such as hybrid reactors that have a dense bed or fixed bed reaction zones and/or fast fluidized bed reaction zones coupled together, circulating fluidized bed reactors, riser reactors, and the like. Suitable conventional reactor types are described in for example U.S. Pat. No. 4,076,796, U.S. Pat. No. 6,287,522 (dual riser), and Fluidization Engineering, D. Kunii and O. Levenspiel, Robert E. Krieger Publishing Company, New York, N.Y. 1977, which are all herein fully incorporated by reference.

The preferred reactor type are riser reactors generally described in Riser Reactor, Fluidization and Fluid-Particle Systems, pages 48 to 59, F. A. Zenz and D. F. Othmer, Reinhold Publishing Corporation, New York, 1960, and U.S. Pat. No. 6,166,282 (fast-fluidized bed reactor), and U.S. patent application Ser. No. 09/564,613 filed May 4, 2000 (multiple riser reactor), which are all herein fully incorporated by reference.

In an embodiment, the amount of liquid feedstock fed separately or jointly with a vapor feedstock, to a reactor system is in the range of from 0.1 weight percent to about 85 weight percent, preferably from about 1 weight percent to about 75 weight percent, more preferably from about 5 weight percent to about 65 weight percent based on the total weight of the feedstock including any diluent contained therein. The liquid and vapor feedstocks are preferably the same composition, or contain varying proportions of the same or different feedstock with the same or different diluent.

The conversion temperature employed in the conversion process, specifically within the reactor system, is in the range of from about 392° F. (200° C.) to about 1832° F. (1000° C.), preferably from about 482° F. (250° C.) to about 1472° F. (800° C.), more preferably from about 482° F. (250° C.) to about 1382° F. (750° C.), yet more preferably from about 572° F. (300° C.) to about 1202° F. (650° C.), yet even more preferably from about 662° F. (350° C.) to about 1112° F. (600° C.) most preferably from about 662° F. (350° C.) to about 1022° F. (550° C.).

The conversion pressure employed in the conversion process, specifically within the reactor system, varies over a wide range including autogenous pressure. The conversion pressure is based on the partial pressure of the feedstock exclusive of any diluent therein. Typically the conversion pressure employed in the process is in the range of from about 0.1 kPaa to about 5 MPaa, preferably from about 5 kPaa to about 1 MPaa, and most preferably from about 20 kPaa to about 500 kPaa.

The weight hourly space velocity (WHSV), particularly in a process for converting a feedstock containing one or more oxygenates in the presence of a molecular sieve catalyst composition within a reaction zone, is defined as the total weight of the feedstock excluding any diluents to the reaction zone per hour per weight of molecular sieve in the molecular sieve catalyst composition in the reaction zone. The WHSV is maintained at a level sufficient to keep the catalyst composition in a fluidized state within a reactor.

Typically, the WHSV ranges from about 1 hr⁻¹ to about 5000 hr⁻¹, preferably from about 2 hr⁻¹ to about 3000 hr⁻¹, more preferably from about 5 hr⁻¹ to about 1500 hr⁻¹, and most preferably from about 10 hr⁻¹ to about 1000 hr⁻¹. In one preferred embodiment, the WHSV is greater than 20 hr⁻¹, preferably the WHSV for conversion of a feedstock containing methanol, DME, or both, is in the range of from about 20 hr⁻¹ to about 300 hr⁻¹.

The superficial gas velocity (SGV) of the feedstock including diluent and reaction products within the reactor system is preferably sufficient to fluidize the molecular sieve catalyst composition within a reaction zone in the reactor. The SGV in the process, particularly within the reactor system, more particularly within the riser reactor(s), is at least 0.1 meter per second (m/sec), preferably greater than 0.5 m/sec, more preferably greater than 1 m/sec, even more preferably greater than 2 m/sec, yet even more preferably greater than 3 m/sec, and most preferably greater than 4 m/sec. See for example U.S. patent application Ser. No. 09/708,753 filed Nov. 8, 2000, which is herein incorporated by reference.

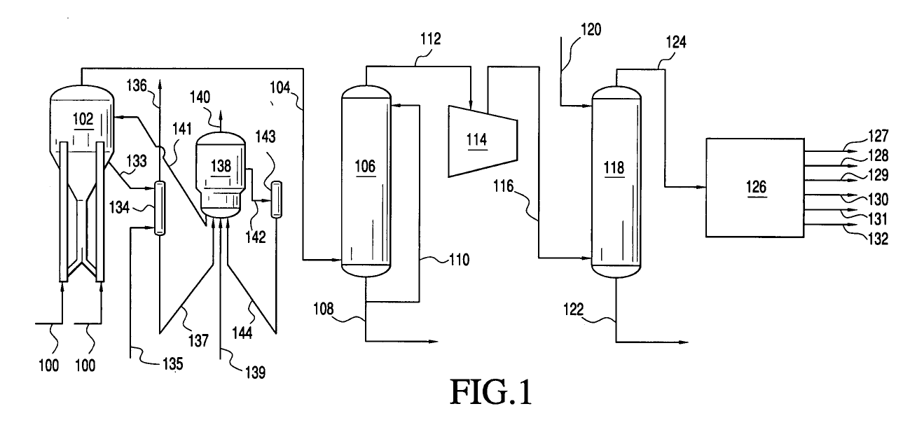

FIG. 1 illustrates a non-limiting exemplary OTO reaction system. In the figure, an oxygenate-containing feedstock is directed through lines 100 to an OTO fluidized reactor 102 wherein the oxygenate (preferably comprising methanol) in the oxygenate-containing feedstock contacts a molecular sieve catalyst composition under conditions effective to convert the oxygenate to light olefins and various byproducts, which are yielded from the fluidized reactor 102 in an olefin-containing stream in line 104. The olefin-containing stream in line 104 optionally comprises methane, ethylene, ethane, propylene, propane, various oxygenate byproducts, C4+ olefins, water and hydrocarbon components. The olefin-containing stream in line 104 is directed to a quench unit or quench tower 106 wherein the olefin-containing stream in line 104 is cooled and water and other readily condensable components are condensed.

The condensed components, which comprise water, are withdrawn from the quench tower 106 through a bottoms line 108. A portion of the condensed components are recycled through line 110 back to the top of the quench tower 106. The components in line 110 preferably are cooled in a cooling unit, e.g., heat exchanger (not shown), so as to provide a cooling medium to cool the components in quench tower 106.

An olefin-containing vapor is yielded from the quench tower 106 through overhead stream 112. The olefin-containing vapor is compressed in one or more compressors 114 and the resulting compressed olefin-containing stream is optionally passed through line 116 to a water absorption unit 118. Methanol is preferably used as the water absorbent, and is fed to the top portion of the water absorption unit 118 through line 120. Methanol and entrained water, as well as some oxygenates, are separated as a bottoms stream through line 122. The light olefins are recovered through an overhead effluent stream 124, which comprises light olefins. Optionally, the effluent stream 124 is sent to an additional compressor or compressors, not shown, and a heat exchanger, not shown. Ultimately, the effluent stream 124 is directed to separation system 126, which optionally comprises one or more separation units such as CO₂ removal unit(s) (e.g., caustic tower(s)), distillation columns, absorption units, and/or adsorption units.

The separation system 126 separates the components contained in the overhead line 124. Thus, separation system 126 forms a light ends stream 127, optionally comprising methane, hydrogen and/or carbon monoxide; an ethylene-containing stream 128 comprising mostly ethylene; an ethane-containing stream 129 comprising mostly ethane; a propylene-containing stream 130 comprising mostly propylene; a propane-containing stream 131 comprising mostly propane; and one or more byproduct streams, shown as line 132, comprising one or more of the oxygenate byproducts, provided above, heavy olefins, heavy paraffins, and/or absorption mediums utilized in the separation process. Separation processes that may be utilized to form these streams are well-known and are described, for example, in pending U.S. patent application Ser. No. 10/124,859 filed Apr. 18, 2002; Ser. No. 10/125,138 filed Apr. 18, 2002; Ser. No. 10/383,204 filed Mar. 6, 2003; and Ser. No. 10/635,410 filed Aug. 6, 2003, the entireties of which are incorporated herein by reference.

FIG. 1 also illustrates a catalyst regeneration system, which is in fluid communication with fluidized reactor 102. As shown, at least a portion of the catalyst compositions contained in fluidized reactor 102 are withdrawn and transported, preferably in a fluidized manner, in conduit 133 from the fluidized reactor 102 to a catalyst stripper 134. In the catalyst stripper 134, the catalyst compositions contact a stripping medium, e.g., steam and/or nitrogen, under conditions effective to remove interstitial hydrocarbons from the molecular sieve catalyst compositions. As shown, stripping medium is introduced into catalyst stripper 134 through line 135, and the resulting stripped stream 136 is released from catalyst stripper 134. Optionally, all or a portion of stripped stream 136 is directed back to fluidized reactor 102.

During contacting of the oxygenate feedstock with the molecular sieve catalyst composition in the fluidized reactor 102, the molecular sieve catalyst composition may become at least partially deactivated. That is, the molecular sieve catalyst composition becomes at least partially coked. In order to reactivate the molecular sieve catalyst composition, the catalyst composition preferably is directed to a catalyst regenerator 138. As shown, the stripped catalyst composition is transported, preferably in the fluidized manner, from catalyst stripper 134 to catalyst regenerator 138 in conduit 137.

In catalyst regenerator 138, the stripped catalyst composition contacts a regeneration medium, preferably comprising oxygen, under conditions effective (preferably including heating the coked catalyst) to at least partially regenerate the catalyst composition contained therein. As shown, the regeneration medium is introduced into the catalyst regenerator 138 through line 139, and the resulting regenerated catalyst compositions are ultimately transported, preferably in a fluidized manner, from catalyst regenerator 138 back to the fluidized reactor 102 through conduit 141. The gaseous combustion products are released from the catalyst regenerator 138 through flue gas stream 140. In another embodiment, not shown, the regenerated catalyst composition additionally or alternatively is directed, optionally in a fluidized manner, from the catalyst regenerator 138 to one or more of the fluidized reactor 102 and/or the catalyst stripper 134. In one embodiment, not shown, a portion of the catalyst composition in the reaction system is transported directly, e.g., without first passing through the catalyst stripper 134, optionally in a fluidized manner, from the fluidized reactor 102 to the catalyst regenerator 138.

As the catalyst compositions contact the regeneration medium in catalyst regenerator 138, the temperature of the catalyst composition will increase due to the exothermic nature of the regeneration process. As a result, it is desirable to control the temperature of the catalyst composition by directing at least a portion of the catalyst composition from the catalyst regenerator 138 to a catalyst cooler 143. As shown, the catalyst composition is transported in a fluidized manner from catalyst regenerator 138 to the catalyst cooler 143 through conduit 142. The resulting cooled catalyst composition is transported, preferably in a fluidized manner, from catalyst cooler 143 back to the catalyst regenerator 138 through conduit 144. In another embodiment, not shown, the cooled catalyst composition additionally or alternatively is directed, optionally in a fluidized manner, from the catalyst cooler 143 to one or more of the fluidized reactor 102 and/or the catalyst stripper 134.

C. Ethanol to Ethylene Reaction Processes

As indicated above, one aspect of the invention is directed to converting ethanol to ethylene. The ethanol to ethylene (ETE) reaction process will now be described in greater detail.

In an ETE reaction system, ethanol in an ethanol-containing feedstock contacts an ETE catalyst composition under conditions effective to convert the ethanol to ethylene and water. Ideally, the catalyst composition comprises a silica-alumina catalyst composition. Silica-alumina catalysts are particularly desirable in such conversion processes, because they are highly selective in the formation of ethylene. Optionally, the ETE catalyst composition is selected from the group consisting of: silica-alumina, alumina (including activated alumina), activated clays, solid phosphoric acid, and a metal sufate. Optionally, the ETE catalyst composition comprises a metal oxide selected from the group consisting of: SiO₂, ThO₂, Al₂O₃, W₂O₄, and Cr₂O₃.

Optionally, the catalyst composition comprises a crystalline aluminosilicate zeolite type of natural or synthetic origin, as described, for example, in U.S. Pat. No. 4,727,214, the entirety of which is incorporated herein by reference. Optionally, the catalyst composition comprises an activated alumina catalyst containing one or more of: an alkali metal, sulfur, iron and/or silicon, as described in U.S. Pat. No. 4,302,357, the entirety of which is incorporated herein by reference. Optionally, the catalyst composition comprises a ZSM-5 and/or a ZSM-11 catalyst composition as described in U.S. Pat. No. 4,698,452, the entirety of which is incorporated herein by reference. In another embodiment, the catalyst composition comprises a substituted phosphoric acid catalyst, as described in U.S. Pat. No. 4,423,270, the entirety of which is incorporated herein by reference. Other potential ethanol to ethylene catalyst compositions that may be implemented in the present invention include, but are not limited to, alumina and magnesia deposited on a porous silica carrier (Haggin, C & EN, May 18, 1981, pp. 52-54), Bauxite activated with phosphoric acid (Chem. Abst., 91, 12305 (1979)), SynDol (N. K. Kochar, R. Merims, and A. S. Padia, Chem. Eng. Progr., June, 1981, 77, 66-70), and polyphosphoric acid, (Pearson et al., Ind. Eng. Chem. Prod. Res. Dev., 19, 245-250 (1980)).

In a conventional ETE reaction process, the ethanol-containing feedstock comprises greater than about 90 weight percent ethanol, more preferably greater than about 95 weight percent ethanol, and most preferably greater than 98 weight percent ethanol, based on the total weight of the ethanol-containing feedstock (although the feedstock according to the present invention preferably contains much lower amounts of ethanol). Optionally, the ETE feedstock further comprises one or more organic compounds containing at least one oxygen atom in addition to ethanol. For example, the oxygenate in the feedstock optionally comprises, in addition to ethanol, one or more other alcohols, preferably aliphatic alcohols where the aliphatic moiety of the alcohol(s) has from 1 to 20 carbon atoms, preferably from 1 to 10 carbon atoms, and most preferably from 1 to 4 carbon atoms. The alcohols useful as feedstock in addition to the ethanol in the process of the invention include lower straight and branched chain aliphatic alcohols and their unsaturated counterparts. Non-limiting examples of possible oxygenates (in addition to ethanol) that may be included in the ETE feedstock include methanol, n-propanol, isopropanol, methyl ethyl ether, DME, diethyl ether, di-isopropyl ether, formaldehyde, dimethyl carbonate, dimethyl ketone, acetic acid, and mixtures thereof. The ETE feedstock also optionally comprises a minor amount of acetaldehyde.

The various feedstocks discussed above are converted primarily into one or more olefins. The olefins or olefin monomers produced from the feedstock typically have from 2 to 30 carbon atoms, preferably 2 to 8 carbon atoms, more preferably 2 to 6 carbon atoms, still more preferably 2 to 4 carbons atoms, and most preferably ethylene and/or propylene. In conventional ETE reaction processes, the catalyst composition utilized to convert the ethanol in the ethanol-containing feedstock to ethylene has a very high conversion and selectivity for ethylene. Typically, the conversion is on the order of greater than about 70, greater than about 90, or greater than about 95 weight percent. The selectivity for ethylene preferably is greater than about 80, greater than about 90, or greater than about 95 weight percent.

In a preferred embodiment, the feedstock, which ideally comprises ethanol, is converted in the presence of a silica-alumina catalyst composition into olefin(s) having 2 to 6 carbons atoms, preferably 2 to 4 carbon atoms, and most preferably ethylene.

The ethanol-containing feedstock, in one embodiment, contains one or more diluents, typically used to reduce the concentration of the feedstock. The diluents are generally non-reactive to the feedstock or the silica-alumina catalyst composition. Non-limiting examples of diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred. In other embodiments, the feedstock does not contain any diluent.

The diluent may be used either in a liquid or a vapor form, or a combination thereof. The diluent is either added directly to a feedstock entering into a reactor or added directly into a reactor, or added with a molecular sieve catalyst composition. In one embodiment, the amount of diluent in the feedstock is in the range of from about 1 to about 99 mole percent based on the total number of moles of the feedstock and diluent, preferably from about 1 to 80 mole percent, more preferably from about 5 to about 50, most preferably from about 5 to about 25. In one embodiment, other hydrocarbons are added to a feedstock either directly or indirectly, and include olefin(s), paraffin(s), aromatic(s) (see for example U.S. Pat. No. 4,677,242, addition of aromatics) or mixtures thereof, preferably propylene, butylene, pentylene, and other hydrocarbons having 4 or more carbon atoms, or mixtures thereof.

The process for converting a feedstock, especially a feedstock containing ethanol, in the presence of a silica-alumina catalyst composition of the invention, is carried out in a reaction process in a reactor, where the process is a fixed bed process or a fluidized bed process (includes a turbulent bed process), preferably a continuous fluidized bed process. Optionally, the reaction process is a fast-fluidized reaction process.

The ETE reaction process can take place in a variety of catalytic reactors such as hybrid reactors that have a dense bed or fixed bed reaction zones and/or fast fluidized bed reaction zones coupled together, circulating fluidized bed reactors, riser reactors, a reactive distillation column, and the like. Suitable conventional reactor types are described in for example Fluidization Engineering, D. Kunii and O. Levenspiel, Robert E. Krieger Publishing Company, New York, N.Y. 1977, which are all herein fully incorporated by reference. Optionally, the ETE reaction process occurs in a tubular reactor or a multi-bed stage reactor (e.g., with more than one bed per vessel) optionally with interbed reheat.

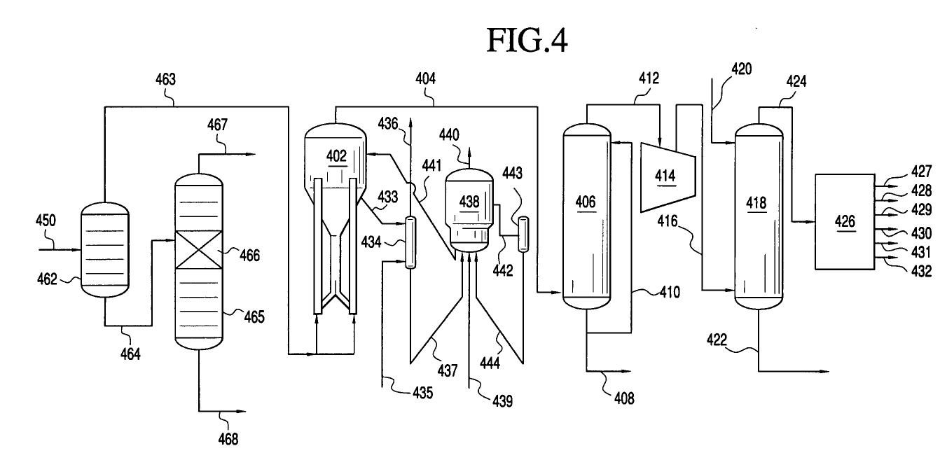

In one embodiment, the amount of liquid feedstock is vaporized and preheated before entering the reactor. The feed is preferable heated to about 220 to 350° C. The conversion temperature employed in the ETE conversion process preferably is significantly lower than in MTO conversion processes. The conversion temperature preferably is in the range of from about 680° F. (360° C.) to about 750° F. (399° C.). The ETE conversion temperature preferably is in the range of from about 150° C. to about 400° C. if the ETE reaction process occurs in a reactive distillation column, as discussed below with reference to FIG. 4.

The conversion pressure employed in the ETE conversion process, specifically within the reactor system, varies over a wide range including autogenous pressure. The conversion pressure is based on the partial pressure of the feedstock exclusive of any diluent therein. Typically the conversion pressure employed in the process is in the range of from about 0.1 kPaa to about 5 MPaa, preferably from about 5 kPaa to about 1 MPaa, and most preferably from about 10 kPaa to about 500 kPaa.

The weight hourly space velocity (WHSV), particularly in a process for converting a feedstock containing ethanol in the presence of a silica-alumina catalyst composition within a reaction zone, is defined as the total weight of the ethanol excluding any diluents to the reaction zone per hour per weight of silica-almina catalyst composition in the reaction zone. Typically, the WHSV ranges from about 0.1 hr⁻¹ to about 0.9 hr⁻¹.

The superficial gas velocity (SGV) of the feedstock including diluent and reaction products within the fluid reactor system is preferably sufficient to fluidize the silica-alumina catalyst composition within a reaction zone in the reactor. The SGV in the process, particularly within the reactor system, is at least 0.5 feet per second (ft/sec) (0.152 m/s), preferably greater than 0.8 ft/sec (0.244 m/s).

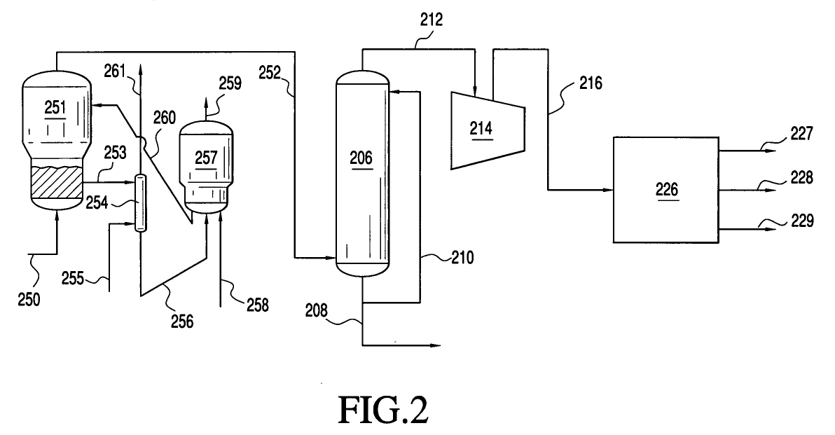

FIG. 2 illustrates a non-limiting exemplary ETE reaction system. In the figure, an ethanol-containing feedstock is directed through line 250 to an ETE reactor 251, which preferably is a fixed bed, a fluidized reactor (as shown) or a fast-fluidized bed reactor, wherein the ethanol in the ethanol-containing feedstock 250 contacts a catalyst composition, preferably a silica-alumina catalyst composition, under conditions effective to convert the ethanol to ethylene and various byproducts, which are yielded from the reactor 251 in an olefin-containing stream in line 252. The olefin-containing stream in line 252 optionally comprises carbon dioxide, methane, ethylene, ethane, propane, butane, various oxygenate byproducts and water. The olefin-containing stream in line 252 is directed to a quench unit or quench tower 206 wherein the olefin-containing stream in line 252 is cooled and water and other readily condensable components are condensed.

The condensed components, which comprise water, are withdrawn from the quench tower 206 through a bottoms line 208. A portion of the condensed components are recycled through line 210 back to the top of the quench tower 206. The components in line 210 preferably are cooled in a cooling unit, e.g., heat exchanger (not shown), so as to provide a cooling medium to cool the components in quench tower 206.

An olefin-containing vapor is yielded from the quench tower 206 through overhead stream 212. The olefin-containing vapor is compressed in one or more compressors 214 and the resulting compressed olefin-containing stream is optionally passed through line 216 to a separation system 226, which optionally comprises one or more separation units such as absorption units, adsorption units and/or distillation columns.

The separation system 226 separates the components contained in the line 216. Thus, separation system 226 forms a light ends stream 227, optionally comprising methane, hydrogen and/or carbon monoxide, an ethylene-containing stream 228 comprising mostly ethylene, and a fuel stream 229 comprising mostly ethane, propane, butane and other oxygenated hydrocarbon byproducts.

FIG. 2 also illustrates a catalyst regeneration system, which is in fluid communication with reactor 251. As shown, at least a portion of the catalyst composition contained in reactor 251 is withdrawn and transported, preferably in a fluidized manner, in conduit 253 from the reactor 251 to a catalyst stripper 254. In the catalyst stripper 254, the catalyst composition contacts a stripping medium, e.g., steam and/or nitrogen, under conditions effective to remove interstitial hydrocarbons from the catalyst composition. As shown, stripping medium is introduced into catalyst stripper 254 through line 255, and the resulting stripped stream 261 is released from catalyst stripper 254. Optionally, all or a portion of stripped stream 261 is directed back to reactor 251.

During contacting of the ethanol-containing feedstock with the dehydration catalyst, preferably silica-alumina, in the reactor 251, the catalyst may become at least partially deactivated. That is, the catalyst becomes at least partially coked. In order to reactivate the catalyst, the catalyst preferably is directed to a catalyst regenerator (in a fluidized bed ETE reaction system) or the reactor is taken off-line for catalyst regeneration (in a fixed bed ETE reaction system). In the fluidized bed ETE reaction system shown, the catalyst composition preferably is directed to a catalyst regenerator 257 in order to reactivate the catalyst. As shown, the stripped catalyst composition is transported, preferably in the fluidized manner, from catalyst stripper 254 to catalyst regenerator 257 in conduit 256.

In the fluidized bed reactor embodiment shown, the catalyst regenerator 257 utilizes an oxygen rich medium, such as air, to regenerate or at least partially regenerate the catalyst composition contained therein. As shown, the regeneration medium is introduced into the catalyst regenerator 257 through line 258, and the resulting regenerated catalyst compositions are ultimately transported, preferably in a fluidized manner, from catalyst regenerator 257 back to the fluidized reactor 251 through conduit 260. The gaseous combustion products are released from the catalyst regenerator 257 through flue gas stream 259.

Optionally, a portion of the catalyst particles in catalyst regenerator 257 are withdrawn and directed to a catalyst cooler, not shown, to control the temperature of the catalyst contained in catalyst regenerator 257. In the catalyst cooler, the catalyst particles indirectly contact a cooling medium, e.g., water and/or steam, under conditions effective to cool the catalyst particles to form cooled catalyst particles, which are directed back to the catalyst regenerator 257 and/or to reactor 251.

In the fixed bed reactor embodiment, not shown, the catalyst preferably is regenerated off-line. The fixed bed reactor comprises at least two, preferable three catalyst beds. In this aspect of the invention, one or more catalyst beds are in service while the other(s) are being regenerated.

D. Combined Methanol/Ethanol to Light Olefins Reaction Processes

As discussed above, the present invention is directed to processes for converting a mixed alcohol-containing feedstock, preferably comprising both methanol and ethanol, to light olefins while minimizing the formation of undesirable byproducts such as acetaldehyde. There are three principal embodiments of this invention. In the first embodiment, the mixed alcohol-containing feed is directed to an ETE reactor for the conversion of ethanol to ethylene, and the resulting effluent stream is then directed to a MTO reactor for the conversion of the methanol in the effluent stream to additional light olefins. In the second embodiment, the methanol and ethanol in the mixed alcohol-containing stream are separated from one another in a separation unit, and the resulting streams are directed to separate ETE and MTO reactors, which operate in parallel. The resulting effluent streams preferably are combined to form a combined stream, which is directed to a single separation system for the separation of the various components contained therein. In the third embodiment, the methanol and ethanol in the mixed alcohol-containing stream are directed to a single reactor, in which the methanol and ethanol contact a mixture of MTO catalyst particles and ETE catalyst particles under conditions effective to convert the methanol to light olefins and the ethanol to light olefins.

The precise composition of the feedstock may vary widely, so long as it contains some methanol and some ethanol. In one embodiment, the weight ratio of methanol to ethanol in the feedstock is greater than 5.0 and less than 49.0, more preferably greater than 6.0 and less than 10.0, even more preferably greater than 6.5 and less than 9.5, with 7.3 being particularly preferred. In terms of weight percent ethanol, the feedstock preferably comprises greater than 1.0 and less than 20.0 weight percent ethanol, more preferably greater than 9.1 and less than 14.2 weight percent ethanol, even more preferably greater than 9.5 and less than 13.3 weight percent ethanol, and most preferably about 12 weight percent ethanol, the balance preferably substantially being methanol. In another embodiment, the methanol to ethanol weight ratio in the feedstock is from about 1 to about 100, optionally from about 3 to about 20. Optionally, the feedstock further comprises greater than about 1 weight percent or greater than about 10 weight percent water, based on the total weight of the feedstock. Optionally, the feedstock further comprises one or more C3+ alcohols, for example, on the order of greater than about 1 weight percent, greater than about 2 weight percent or greater than about 4 weight percent C3+ alcohols, based on the total weight of the feedstock. Ideal feedstocks for the present invention are described in U.S. patent application Ser. Nos. 10/716,685; 10/716,894; 10/717,006 and in PCT Application No. PCT/US2004/035474, previously incorporated by reference.

It is noted, however, that the present invention is not limited to converting methanol and ethanol in the above-described ratios to light olefins. For example, it is also contemplated by the present invention that the weight ratio of methanol to ethanol contained in the feedstock may deviate from the preferred ratios provided above. Ethanol exhibits a greater selectively to ethylene than does methanol, which typically converts to ethylene and propylene in equal amounts. Accordingly, by controlling the weight ratio of the methanol to ethanol that is directed to the OTO reaction system of the present invention, the weight ratio of ethylene to propylene formed in the OTO reaction system can be desirably controlled in response, for example, to fluctuations in commercial market conditions for ethylene and propylene.

In other words, the present invention provides the ability to produce more ethylene relative to propylene (ethylene is typically more valuable and/or in greater demand than propylene) than in conventional OTO reaction systems. For example, a typical MTO reaction system, which receives a feedstock in which the only reactive species is methanol, typically forms light olefins having a weight ratio of ethylene to propylene of from about 0.95 to about 0.98. Changes in reaction conditions, e.g., temperature and pressure, may impact percent conversion in the MTO reaction system, but typically will not have a dramatic effect on overall ethylene and propylene selectivities. In contrast, according to one aspect of the present invention, the overall amount of ethylene formed in an OTO reaction system of the present invention can be advantageously increased relative to propylene formed. The light olefins formed according to the present invention may have a weight ratio of ethylene to propylene of greater than about 0.7, greater than about 1.0, greater than about 1.2, greater than about 1.5, or greater than about 2.0. Preferably, however, the ethylene to propylene weight ratio ranges from about 0.8 to about 2.5, more preferably from about 1.0 to about 2.0, and most preferably from about 1.0 to about 1.2. A weight ratio of from about 1.0 to about 1.2 is particularly preferred because this ratio of ethylene to propylene generally corresponds with current commercial demands for these commodity olefins. These weight ratios are based on the total amount of light olefins formed in the overall reaction system, whether it is a two step reaction process or a single step reaction process, as discussed in more detail below.

In addition to providing the ability to synthesize light olefins at a desirable prime olefin ratio, the effluent formed in an OTO reaction system of the present invention comprises a low level of undesirable contaminants. In particular, the production of acetaldehyde, which may be difficult to separate from a reaction effluent, is advantageously minimized according to the present invention. Additionally, the amount of aromatic compounds, which can poison polymerization catalysts, has been a problem of conventional OTO conversion processes, particularly conversion processes implementing ZSM-5 and/or modified ZSM-5 catalyst compositions. See, e.g., U.S. Pat. No. 4,698,452, issued Oct. 6, 1987, the entirety of which is incorporated herein by reference.

For example, the first effluent and/or the second effluent yielded from the first and second reactors, respectively, preferably comprise less than 2 weight percent, more preferably less than about 1 weight percent, and most preferably less than 0.2 weight percent acetaldehyde, based on the total weight of the respective effluent stream. In the single step reaction process described below, the effluent stream also preferably comprises less than about 2 weight percent, more preferably less than about 1 weight percent, and most preferably less than 0.2 weight percent acetaldehyde, based on the total weight of the effluent stream.

The process of the present invention additionally has the ability of forming an effluent stream comprising little if any aromatic components. In one embodiment, the first effluent and/or the second effluent (or the effluent from the single step reaction process) comprises less than 5.0 weight percent, more preferably less than 1.0 weight percent, and more preferably less than 0.05 weight percent aromatic compounds, based on the total weight of the respective effluent stream. Such low levels of aromatic components can be realized if the MTO conversion catalyst comprises a molecular sieve selected from the group consisting of: MeAPSO, SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, AEI/CHA intergrowths, metal containing forms thereof, intergrown forms thereof, and mixtures thereof, SAPO-34, AEI/CHA intergrowths being particularly preferred. In another embodiment, the MTO and ETE catalyst compositions implemented in converting the methanol and ethanol to light olefins does not comprise (excludes) a ZSM-5 or modified ZSM-5 catalyst composition.

1. Converting a Mixed Feedstock to Light Olefins with Two Reactors Operating in Series

As indicated above, in one embodiment, the invention is to a process for producing light olefins, the process comprising the steps of: (a) providing a feedstock comprising methanol and ethanol; (b) dehydrating at least a portion of the ethanol in a first reactor to form a first effluent comprising ethylene, methanol, water and less than about 2 weight percent acetaldehyde, based on the total weight of the first effluent; and (c) contacting the methanol in the first effluent with a molecular sieve catalyst composition in a second reactor under conditions effective to convert the methanol to additional light olefins. Optionally, the process further comprises the step of: (d) removing a weight majority of the water from the first effluent between steps (b) and (c).

In this embodiment, step (b) preferably comprises contacting the ethanol with a dehydration catalyst under conditions effective to convert the ethanol to the ethylene and water, wherein the dehydration catalyst is selected from the group consisting of: silica-alumina, activated alumina, phosphoric acid, and activated clay. For purposes of the present invention, the terms “dehydration catalyst” and “ETE catalyst” are synonymous and interchangeably used herein.

The composition of the first effluent may vary depending, for example, on the amount of ethanol in the feedstock, the dehydration catalyst used, and reaction conditions. In one embodiment, the first effluent comprises less than about 2 weight percent, less than about 1 weight percent, less than about 0.2 weight percent, less than about 0.1 weight percent, or less than about 0.05 weight percent acetaldehyde, based on the total weight of the first effluent. Additionally or alternatively, the first effluent optionally comprises at least about 5, or at least about 10 weight percent ethylene, based on the total weight of the first effluent. Additionally or alternatively, the first effluent comprises carbon dioxide, methane, ethylene, ethane, propylene, propane, acetaldehyde, butane, diethyl ether, water, methanol and/or dimethyl ether. Ethanol products other than ethylene and acetaldehyde preferably are at trace levels, although it is contemplated that some poorer ETE catalyst compositions may convert as much as 20 wt. percent of the feed carbon into acetaldehyde consistent with the above-disclosed lower ETE selectivity levels.

Preferably, weight majority of the methanol from the feedstock passes through the first reactor and into the first effluent, although it is contemplated that a portion of the methanol may be converted to dimethyl ether (DME) and/or light olefins in the first reactor. Thus, the first effluent optionally comprises at least about 5, at least about 15, or at least about 25 weight percent methanol, based on the total weight of the first effluent. At least a portion of the methanol from the feedstock optionally is dehydrated in the first reactor to DME. In this embodiment, the first effluent optionally further comprises the DME. In this aspect of the present invention, the first effluent optionally comprises at least about 5 weight percent or at least about 25 weight percent DME, based on the total weight of the first effluent. Optionally, the process further comprises the step of: contacting at least a portion of the DME with the molecular sieve catalyst composition in the second reactor under conditions effective to convert the DME to ethylene.

In one aspect of the invention, discussed in detail below with reference to FIG. 4, the first reactor comprises an alcohol dehydration reactive distillation column. In this aspect of the invention, a weight majority of the water formed in step (b) optionally is separated in the distillation column from a weight majority of the methanol and ethylene, collectively, formed in step (b). Alternatively, the first reactor comprises a fixed bed reactor, a fluidized bed reactor or a fast-fluidized reactor.

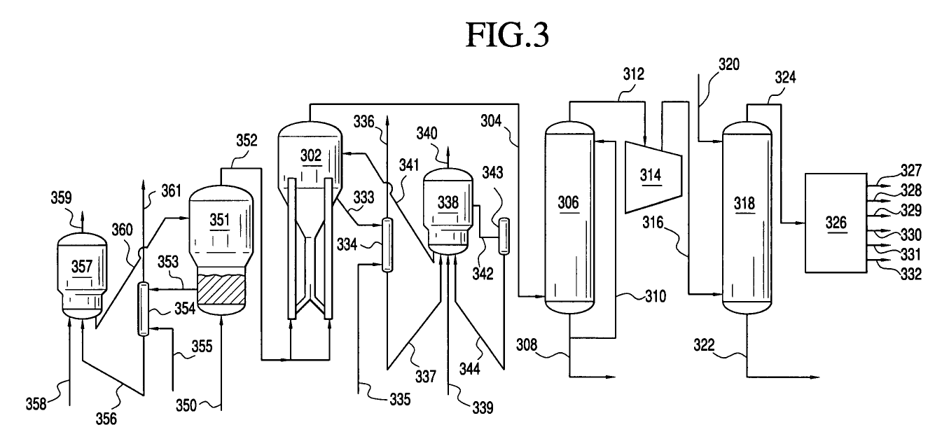

FIG. 3 illustrates one non-limiting embodiment of this aspect of the present invention. As shown, a feedstock 350 comprising methanol and ethanol is introduced into a first reactor 351. The first reactor 351 preferably comprises a fixed bed reactor, a fluidized bed reactor (as shown) or a fast-fluidized bed reactor. In the first reactor 351, the ethanol in the feedstock 350 contacts a catalyst composition, preferably a silica-alumina catalyst composition, under conditions effective to convert the ethanol to ethylene and various byproducts, which are yielded from the first reactor 351 in a first effluent 352. That is, in first reactor 351, at least a portion of the ethanol in the first reactor is dehydrated to form the first effluent 352, which comprises ethylene, methanol, water and less than about 2 weight percent acetaldehyde, based on the total weight of the first effluent.

As shown, the first effluent 352, preferably is directed to a second reactor 302. Second reactor 302 preferably comprises a fluidized bed reactor or a fast-fluidized reactor (as shown). In second reactor 302, the methanol from first effluent 352 preferably contacts a molecular sieve catalyst composition under conditions effective to convert the methanol to light olefins and various byproducts, which are yielded from the second reactor 302 in second effluent 304. The second effluent 304 optionally comprises methane, ethylene, ethane, propylene, propane, various oxygenate byproducts, C4+ olefins, water and hydrocarbon components. The second effluent 304 is directed to a quench unit or quench tower 306 wherein the second effluent 304 is cooled and water and other readily condensable components are condensed.

The condensed components, which comprise water, are withdrawn from the quench tower 306 through a bottoms line 308. A portion of the condensed components are recycled through line 310 back to the top of the quench tower 306. The components in line 310 preferably are cooled in a cooling unit, e.g., heat exchanger (not shown), so as to provide a cooling medium to cool the components in quench tower 306.

An olefin containing vapor is yielded from the quench tower 306 through overhead stream 312. The olefin containing vapor is compressed in one or more compressors 314 and the resulting compressed olefin containing stream is optionally passed through line 316 to a water absorption unit 318. Methanol is preferably used as the water absorbent, and is fed to the top portion of the water absorption unit 318 through line 320. Methanol and entrained water, as well as some oxygenates, are separated as a bottoms stream through line 322. The light olefins are recovered through an overhead effluent stream 324, which comprises light olefins. Optionally, the effluent stream 324 is sent to an additional compressor or compressors, not shown, and a heat exchanger, not shown. Ultimately, the effluent stream 324 is directed to separation system 326, which optionally comprises one or more separation units such as CO₂ removal unit(s) (e.g., caustic tower(s)), distillation columns, absorption units, and/or adsorption units.

The separation system 326 separates the components contained in the overhead effluent stream 324. Thus, separation system 326 forms a light ends stream 327, optionally comprising methane, hydrogen and/or carbon monoxide; an ethylene-containing stream 328 comprising mostly ethylene; an ethane-containing stream 329 comprising mostly ethane; a propylene-containing stream 330 comprising mostly propylene; a propane-containing stream 331 comprising mostly propane; and one or more byproduct streams, shown as line 332, comprising one or more of the oxygenate byproducts, provided above, heavy olefins, heavy paraffins, and/or absorption mediums utilized in the separation process. Separation processes that may be utilized to form these streams are well-known.

FIG. 3 also includes two catalyst regeneration systems. A first catalyst regeneration system is in fluid communication with the first reactor 351, and a second regeneration system is in fluid communication with the second reactor 302. Preferably, the first and second regeneration systems are separated from one another so as to prevent commingling of the catalyst contained in each of the respective catalyst regeneration systems.

In the first catalyst regeneration system, at least a portion of the catalyst composition contained in first reactor 351 is withdrawn and transported, preferably in a fluidized manner, in conduit 353 from the first reactor 351 to a catalyst stripper 354. In the catalyst stripper 354, the catalyst composition contacts a stripping medium, e.g., steam and/or nitrogen, under conditions effective to remove interstitial hydrocarbons from the catalyst composition. As shown, stripping medium is introduced into catalyst stripper 354 through line 355, and the resulting stripped stream 361 is released from catalyst stripper 354. Optionally, all or a portion of the stripped stream 361 is directed back to first reactor 351.

During contacting of the ethanol in feedstock 350 with the alumina catalyst in the first reactor 351, the catalyst may become at least partially deactivated. That is, the catalyst composition becomes at least partially coked. In order to reactivate the catalyst, the catalyst preferably is directed to a catalyst regenerator 357 (in a fluidized bed ETE reaction system as shown) or the reactor is taken off line for catalyst regeneration (in a fixed bed ETE reaction system, not shown). In the fluidized bed ETE reaction system shown, the catalyst composition preferably is directed to a catalyst regenerator 357 in order to reactivate the catalyst. As shown, the stripped catalyst is transported, preferably in a fluidized manner from catalyst stripper 354 to catalyst regenerator 357 in conduit 356.

In the fluidized bed reactor embodiment shown, the catalyst regenerator 357 utilizes an oxygen-rich medium such as air to regenerate or at least partially regenerate the catalyst composition contained therein. As shown, the regeneration medium is introduced into the catalyst regenerator 357 through line 358, and the resulting regenerated catalyst compositions are ultimately transported, preferably in a fluidized manner, from catalyst regenerator 357 back to the first reactor 351 through conduit 360. The gaseous combustion products of the regeneration process are released from the catalyst regenerator 351 through flue gas stream 359.

Optionally, a portion of the catalyst particles in catalyst regenerator 357 are withdrawn and directed to a catalyst cooler, not shown, to control the temperature of the catalyst contained in catalyst regenerator 357. In the catalyst cooler, the catalyst particles indirectly contact a cooling medium, e.g., water and/or steam, under conditions effective to cool the catalyst particles to form cooled catalyst particles, which are directed back to the catalyst regenerator 357 and/or to first reactor 351.

As indicated above, this aspect of the present invention also preferably comprises a second catalyst regeneration system, which is in fluid communication with second reactor 302. As shown, at least a portion of the catalyst compositions contained in second reactor 302 are withdrawn and transported preferably in a fluidized manner in conduit 333 from the second reactor 302 to a catalyst stripper 334. In the catalyst stripper 334, the catalyst compositions contact a stripping medium, e.g., steam and/or nitrogen, under conditions effective to remove interstitial hydrocarbons from the molecular saved catalyst compositions. As shown, stripping medium is introduced into catalyst stripper 334 through line 335, and the resulting stripped stream 336 is released from catalyst stripper 334. Optionally, all or a portion of stripped stream 336 is directed back to second reactor 302.

During contacting of the methanol in first effluent 352 with the molecular sieve catalyst composition in second reactor 302, the molecular sieve catalyst composition may become at least partially deactivated. That is, the molecular sieve catalyst composition becomes at least partially coked. In order to reactivate the molecular sieve catalyst composition, the catalyst composition preferably is directed to a catalyst regenerator 338. As shown, the striped catalyst composition is transported, preferably in a fluidized manner, from catalyst stripper 334 to catalyst regenerator 338 in conduit 337.

In catalyst regenerator 338, the stripped catalyst composition contacts a regeneration medium, preferably comprising oxygen, under conditions effective to at least partially regenerate the catalyst composition contained therein. As shown, the regeneration medium is introduced into the catalyst regenerator 338 through line 339, and the resulting regenerated catalyst compositions are ultimately transported, preferably in a fluidized manner, from catalyst regenerator 338 back to the second reactor 302 through conduit 341. The gaseous combustion products are released from the catalyst regenerator 338 through flue gas stream 340. In another embodiment, not shown, the regenerated catalyst composition additionally or alternatively is directed, optionally in a fluidized manner, from the catalyst regenerator 338 to one or more of the second reactor 302 and/or the catalyst stripper 334. In one embodiment, not shown, a portion of the catalyst composition in the reaction system is transported directly, e.g., without first passing through the catalyst stripper 334, optionally in a fluidized manner, from the second reactor 302 to the catalyst regenerator 338.

As the catalyst compositions contact the regeneration medium in catalyst regenerator 338, the temperature of the catalyst composition will increase due to the exothermic nature of the regeneration process. As a result, it is desirable to control the temperature of the catalyst composition by directing at least a portion of the catalyst composition from the catalyst regenerator 338 to a catalyst cooler 343. As shown, the catalyst composition is transported in the fluidized manner from catalyst regenerator 338 to the catalyst cooler 343 through conduit 342. The resulting cooled catalyst composition is transported, preferably in a fluidized manner, from catalyst cooler 343 back to the catalyst regenerator 338 through conduit 344. In another embodiment, not shown, the cooled catalyst composition additionally or alternatively is directed, optionally in a fluidized manner, from the catalyst cooler 343 to one or more of the second reactor 302 and/or the catalyst stripper 334.

The two-step reaction process described above with reference to FIG. 3, is particularly desirable for converting a feedstock comprising methanol and ethanol to light olefins. It has now been discovered that the dehydration step in the first reactor will facilitate the conversion of ethanol selectively to ethylene, with minimal production of acetaldehyde or other byproducts, as described above. That is, a significant advantage of the present invention is that the ethanol in the feedstock is converted more selectively to desirable ethylene product with little or no production of undesirable byproducts. It also may promote some conversion of methanol in the feedstock to dimethyl ether (DME). However, the conversion of methanol to DME does not pose a problem for the present invention since the resulting DME/methanol mixture in the first effluent would react similarly to methanol alone over a molecular sieve catalyst composition in the second reactor. Additionally, the ethylene in the first effluent beneficially passes through the second reactor without substantially converting to other products.

2. Converting a Mixed Feedstock to Light Olefins with Two Reactors Operating in Parallel

In another embodiment, the invention is to a process for producing light olefins, the process comprising the steps of: (a) providing a feedstock comprising methanol and ethanol; (b) separating the feedstock into a methanol-containing stream and an ethanol-containing stream, wherein the methanol-containing stream comprises a weight majority of the methanol from the feedstock, and the ethanol-containing stream comprises a weight majority of the ethanol from the feedstock; (c) contacting the ethanol in the ethanol-containing stream with a dehydration catalyst in a first reactor under conditions effective to convert the ethanol to water and light olefins, wherein the light olefins are yielded from the first reactor in a first effluent; (d) contacting the methanol in the methanol-containing stream with a molecular sieve catalyst composition in a second reactor under conditions effective to convert the methanol to light olefins and water, which are yielded from the second reactor in a second effluent; and (e) combining at least a portion of the first effluent with at least a portion of the second effluent to form a combined product stream.

Preferably, the methanol-containing stream preferably comprises at least about 60 weight percent, at least 75 weight percent or at least about 90 weight percent of the methanol that was in the feedstock.

In this embodiment, if the feedstock comprises C3+ alcohols, a weight majority of the C3+ alcohols preferably are separated in step (b) into the ethanol-containing stream. The C3+ alcohols preferably also are dehydrated to light olefins and water in the first reactor. In this aspect of the invention, the feedstock optionally comprises more than 1 weight percent C3+ alcohols, based on the weight of the feedstock.

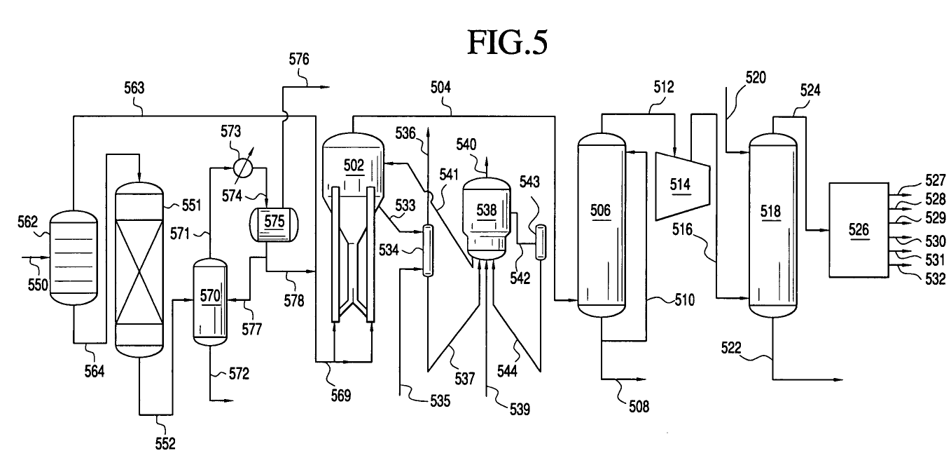

A non-limiting exemplary reaction system in accordance with this embodiment of the present invention is illustrated in FIG. 4. In the figure, a feedstock 450 comprising methanol and ethanol is directed to a separation unit 462. Preferably, the separation unit 462 comprises a rough cut distillation column, which is designed to separate the feedstock 450 into a methanol-containing stream 463 and an ethanol-containing stream 464. The methanol containing stream 463 preferably comprises a weight majority of the methanol from the feedstock 450. The ethanol-containing stream 464 preferably comprises a weight majority of the ethanol from the feedstock 450. As shown, the ethanol-containing stream 464 is directed to first reactor 465. In first reactor 465, the ethanol from ethanol-containing stream 464 contacts the catalyst composition, preferably a dehydration catalyst such as silica alumina, under conditions effective to convert the ethanol to water and light olefins (particularly ethylene). As shown, the first reactor 465 comprises a reactive distillation column. A reactive distillation column is a single unit in which a chemical reaction and distillative separation are carried out simultaneously. Conducting the ETE reaction process in a reactive distillation column is particularly preferred in this embodiment of the present invention in that the water formed in the contacting step can be advantageously separated from the light olefin products formed in the contacting step in a single set of equipment. However, it is contemplated that the first reactor may comprise a fluidized bed reactor, a fast fluidized reactor, or a fixed bed reactor, as shown below with reference to FIG. 5. Reverting to FIG. 4, the light olefins formed in the contacting step preferably are yielded from the first reactor in a first effluent 467. As shown, the first effluent 467 is yielded from the first reactor 465 in an overhead stream. The water formed and the contacting step preferably is yielded from the first reactor 465 (in the reactive distillation column embodiment shown) in water-containing stream 468. As shown, water contained stream 468 comprises a bottoms stream. The catalyst composition used to catalyze the conversion of ethanol to ethylene preferably is situated just below the inlet of the ethanol-containing stream 464 into first reactor 465. It is contemplated that the catalyst composition in reaction zone 466, or a portion thereof, may be regenerated offline as necessary. Optionally, reaction zone 466 comprises at least 2, preferably 3 catalyst beds, and one or more catalyst beds may be in service while the other(s) are being regenerated.

In another embodiment, not shown, the first reactor 465 comprises a fluidized bed reactor, as shown by first reactor 351 in FIG. 3. In this aspect of the present invention, the fluidized bed reactor preferably comprises a regeneration system as shown in FIG. 3.

Reverting to FIG. 4, the methanol-containing stream 463 preferably is directed to second reactor 402 in which methanol (and any ethanol contained in methanol containing stream) in methanol containing stream contacts a molecular sieve catalyst composition under conditions effective to convert the methanol to light olefins and various byproducts, which are yielded from the second reactor 402 in second effluent 404. The second effluent 404 optionally comprises methane, ethylene, ethane, propylene, propane, various oxygenated byproducts, C4+ olefins, water and hydrocarbon components. The second effluent 404 is directed to a quench unit or quench tower 406 wherein the second effluent 404 is cooled and water and other readily condensable components are condensed.

The condensed components, which comprise water, are withdrawn from the quench tower 406 through a bottoms line 408. A portion of the condensed components are recycled through line 410 back to the top of the quench tower 406. The components in line 410 preferably are cooled in a cooling unit, e.g., heat exchanger (not shown), so as to provide a cooling medium to cool the components in quench tower 406.

An olefin-containing vapor is yielded from the quench tower 406 through overhead stream 412. The olefin-containing vapor is compressed in one or more compressors 414 and the resulting compressed olefin-containing stream is optionally passed through line 416 to a water absorption unit 418. Methanol is preferably used as the water absorbent, and is fed to the top portion of the water absorption unit 418 through line 420. Methanol and entrained water, as well as some oxygenates, are separated as a bottoms stream through line 422. The light olefins are recovered through an overhead effluent stream 424, which comprises light olefins. Optionally, the effluent stream 424 is sent to an additional compressor or compressors, not shown, and a heat exchanger, not shown. Ultimately, the effluent stream 424 is directed to separation system 426, which optionally comprises one or more separation units such as CO₂ removal unit(s) (e.g., caustic tower(s)), distillation columns, absorption units, and/or adsorption units.

The separation system 426 separates the components contained in the effluent stream 424. Thus, separation system 426 forms a light ends stream 427, optionally comprising methane, hydrogen and/or carbon monoxide; an ethylene-containing stream 428 comprising mostly ethylene; an ethane-containing stream 429 comprising mostly ethane; a propylene-containing stream 430 comprising mostly propylene; a propane-containing stream 431 comprising mostly propane; and one or more byproduct streams, shown as line 432, comprising one or more of the oxygenate byproducts, provided above, heavy olefins, heavy paraffins, and/or absorption mediums utilized in the separation process. Separation processes that may be utilized to form these streams are well-known and are described, for example, in pending U.S. patent application Ser. No. 10/124,859 filed Apr. 18, 2002; Ser. No. 10/125,138 filed Apr. 18, 2002; Ser. No. 10/383,204 filed Mar. 6, 2003; and Ser. No. 10/635,410 filed Aug. 6, 2003, the entireties of which are incorporated herein by reference.

FIG. 4 also illustrates a catalyst regeneration system, which is in fluid communication with second reactor 402. As shown, at least a portion of the catalyst compositions contained in second reactor 402 are withdrawn and transported, preferably in a fluidized manner, in conduit 433 from the second reactor 402 to a catalyst stripper 434. In the catalyst stripper 434, the catalyst compositions contact a stripping medium, e.g., steam and/or nitrogen, under conditions effective to remove interstitial hydrocarbons from the molecular sieve catalyst compositions. As shown, stripping medium is introduced into catalyst stripper 434 through line 435, and the resulting stripped stream 436 is released from catalyst stripper 434. Optionally, all or a portion of stripped stream 436 is directed back to second reactor 402.

During contacting of the oxygenate feedstock with the molecular sieve catalyst composition in the second reactor 402, the molecular sieve catalyst composition may become at least partially deactivated. That is, the molecular sieve catalyst composition becomes at least partially coked. In order to reactivate the molecular sieve catalyst composition, the catalyst composition preferably is directed to a catalyst regenerator 438. As shown, the stripped catalyst composition is transported, preferably in the fluidized manner, from catalyst stripper 434 to catalyst regenerator 438 in conduit 437.

In catalyst regenerator 438, the stripped catalyst composition contacts a regeneration medium, preferably comprising oxygen, under conditions effective (preferably including heating the coked catalyst) to at least partially regenerate the catalyst composition contained therein. As shown, the regeneration medium is introduced into the catalyst regenerator 438 through line 439, and the resulting regenerated catalyst compositions are ultimately transported, preferably in a fluidized manner, from catalyst regenerator 438 back to the second reactor 402 through conduit 441. The gaseous combustion products are released from the catalyst regenerator 438 through flue gas stream 440. In another embodiment, not shown, the regenerated catalyst composition additionally or alternatively is directed, optionally in a fluidized manner, from the catalyst regenerator 438 to one or more of the second reactor 402 and/or the catalyst stripper 434. In one embodiment, not shown, a portion of the catalyst composition in the reaction system is transported directly, e.g., without first passing through the catalyst stripper 434, optionally in a fluidized manner, from the second reactor 402 to the catalyst regenerator 438.

As the catalyst compositions contact the regeneration medium in catalyst regenerator 438, the temperature of the catalyst composition will increase due to the exothermic nature of the regeneration process. As a result, it is desirable to control the temperature of the catalyst composition by directing at least a portion of the catalyst composition from the catalyst regenerator 438 to a catalyst cooler 443. As shown, the catalyst composition is transported in a fluidized manner from catalyst regenerator 438 to the catalyst cooler 443 through conduit 442. The resulting cooled catalyst composition is transported, preferably in a fluidized manner, from catalyst cooler 443 back to the catalyst regenerator 438 through conduit 444. In another embodiment, not shown, the cooled catalyst composition additionally or alternatively is directed, optionally in a fluidized manner, from the catalyst cooler 443 to one or more of the second reactor 402 and/or the catalyst stripper 434.

In another embodiment, not shown, all or a portion of the first effluent 467 is added to and combined with second effluent 404 to form a combined stream, which is directed to quench tower 406. In another embodiment, not shown, all or a portion of the first effluent 467 is added to and combined with one or more of the overhead stream 412, line 416, and/or overhead effluent stream 424 to form a combined stream, which is ultimately directed to separation system 426.

FIG. 5 illustrates another non-limiting embodiment of this aspect of the present invention. In the figure, a feedstock 550 comprising methanol and ethanol is directed to a separation unit 562. Preferably, the separation unit 562 comprises a rough cut distillation column, which is designed to separate the feedstock 550 into a methanol-containing stream 563 and an ethanol-containing stream 564. The methanol containing stream 563 preferably comprises a weight majority of the methanol from the feedstock 550. The ethanol-containing stream 564 preferably comprises a weight majority of the ethanol from the feedstock 550. As shown, the ethanol-containing stream 564 is directed to first reactor 551. In first reactor 551, the ethanol from ethanol-containing stream 564 contacts a catalyst composition, preferably a dehydration catalyst such as silica-alumina, under conditions effective to convert the ethanol to water and light olefins (particularly ethylene). As shown, the first reactor 551 comprises a fixed bed reactor.

The light olefins (mostly ethylene) and water formed in the contacting step preferably are yielded from the first reactor in a first effluent 552. As shown, the first effluent 552 is yielded from the first reactor 551 and directed to a separation unit 570. Preferably, separation unit 570 comprises one or more distillation columns, although it is contemplated that the separation unit 570 may additionally or alternatively comprise one or more adsorption and/or absorption columns.

As shown, in separation unit 570, the first effluent 552 is subjected to conditions effective to form a water-containing stream 572 and an overhead stream 571. Preferably, the water containing stream 572 comprises a weight majority of the water that was present in the first effluent 552. The overhead stream 571 comprises a weight majority of the light olefins (ethylene and propylene) and methanol that was present in the first effluent 552. In a preferred embodiment, the light olefins and water in overhead stream 571 are separated from one another. As shown, overhead stream 571 is cooled in a heat exchanger 573 to form a cooled overhead stream 574, which is directed to a knockout drum 575 in which readily condensable components are condensed. A liquid fraction comprising a weight majority of the methanol that was contained in overhead stream 571 is removed from the knockout drum 575. A first portion 577 of the liquid fraction preferably is directed back to the separation unit 570 to improve the separation occurring in separation unit 570, and a second portion 578 of the liquid portion is directed to and preferably combined with methanol containing stream 563, as shown, to form combined stream 569. A vapor fraction 576, which preferably comprises a weight majority of the ethylene that was present in the overhead stream 571, also is yielded from knockout drum 575.

The catalyst composition in first reactor 551, or a portion thereof, optionally is regenerated offline as necessary, and as described above. Optionally, first reactor 551 comprises at least 2, preferably 3 catalyst beds, and one or more catalyst beds may be in service while the other(s) are being regenerated.

In another embodiment, not shown, the first reactor 551 comprises a fluidized bed reactor, as shown by first reactor 351 in FIG. 3. In this aspect of the present invention, the fluidized bed reactor preferably comprises a regeneration system as shown in FIG. 3.

Reverting to FIG. 5, the combined stream 569 preferably is directed to second reactor 502 in which methanol (and any ethanol contained in combined stream) in combined stream contacts a molecular sieve catalyst composition under conditions effective to convert the methanol to light olefins and various byproducts, which are yielded from the second reactor 502 in second effluent 504. The second effluent 504 optionally comprises methane, ethylene, ethane, propylene, propane, various oxygenated byproducts, C4+ olefins, water and hydrocarbon components. In a preferred embodiment, all or a portion of vapor fraction 576 is combined with second effluent 504 to form a combined effluent. This embodiment is preferred because it advantageously allows the effluents from the first reactor 551 and the second reactor 502 to share a common separation system.

The second effluent 504, optionally in admixture with vapor stream 576, is directed to a quench unit or quench tower 506 wherein the second effluent 504 is cooled and water and other readily condensable components are condensed. The condensed components, which comprise water, are withdrawn from the quench tower 506 through a bottoms line 508. A portion of the condensed components are recycled through line 510 back to the top of the quench tower 506. The components in line 510 preferably are cooled in a cooling unit, e.g., heat exchanger (not shown), so as to provide a cooling medium to cool the components in quench tower 506.

An olefin-containing vapor is yielded from the quench tower 506 through overhead stream 512. The olefin-containing vapor is compressed in one or more compressors 514 and the resulting compressed olefin-containing stream is optionally passed through line 516 to a water absorption unit 518. Methanol is preferably used as the water absorbent, and is fed to the top portion of the water absorption unit 518 through line 520. Methanol and entrained water, as well as some oxygenates, are separated as a bottoms stream through line 522. The light olefins are recovered through an overhead effluent stream 524, which comprises light olefins. Optionally, the effluent stream 524 is sent to an additional compressor or compressors, not shown, and a heat exchanger, not shown. Ultimately, the effluent stream 524 is directed to separation system 526, which optionally comprises one or more separation units such as CO₂ removal unit(s) (e.g., caustic tower(s)), distillation columns, absorption units, and/or adsorption units).

The separation system 526 separates the components contained in the effluent stream 524. Thus, separation system 526 forms a light ends stream 527, optionally comprising methane, hydrogen and/or carbon monoxide; an ethylene-containing stream 528 comprising mostly ethylene; an ethane-containing stream 529 comprising mostly ethane; a propylene-containing stream 530 comprising mostly propylene; a propane-containing stream 531 comprising mostly propane; and one or more byproduct streams, shown as line 532, comprising one or more of the oxygenate byproducts, provided above, heavy olefins, heavy paraffins, and/or absorption mediums utilized in the separation process. Separation processes that may be utilized to form these streams are well-known and are described, for example, in pending U.S. patent application Ser. No. 10/124,859 filed Apr. 18, 2002; Ser. No. 10/125,138 filed Apr. 18, 2002; Ser. No. 10/383,204 filed Mar. 6, 2003; and Ser. No. 10/635,410 filed Aug. 6, 2003, the entireties of which are incorporated herein by reference.

FIG. 5 also illustrates a catalyst regeneration system, which is in fluid communication with second reactor 502. As shown, at least a portion of the catalyst compositions contained in second reactor 502 are withdrawn and transported, preferably in a fluidized manner, in conduit 533 from the second reactor 502 to a catalyst stripper 534. In the catalyst stripper 534, the catalyst compositions contact a stripping medium, e.g., steam and/or nitrogen, under conditions effective to remove interstitial hydrocarbons from the molecular sieve catalyst compositions. As shown, stripping medium is introduced into catalyst stripper 534 through line 535, and the resulting stripped stream 536 is released from catalyst stripper 534. Optionally, all or a portion of stripped stream 536 is directed back to second reactor 502.

During contacting of the oxygenate feedstock with the molecular sieve catalyst composition in the second reactor 502, the molecular sieve catalyst composition may become at least partially deactivated. That is, the molecular sieve catalyst composition becomes at least partially coked. In order to reactivate the molecular sieve catalyst composition, the catalyst composition preferably is directed to a catalyst regenerator 538. As shown, the stripped catalyst composition is transported, preferably in the fluidized manner, from catalyst stripper 534 to catalyst regenerator 538 in conduit 537.

In catalyst regenerator 538, the stripped catalyst composition contacts a regeneration medium, preferably comprising oxygen, under conditions effective (preferably including heating the coked catalyst) to at least partially regenerate the catalyst composition contained therein. As shown, the regeneration medium is introduced into the catalyst regenerator 538 through line 539, and the resulting regenerated catalyst compositions are ultimately transported, preferably in a fluidized manner, from catalyst regenerator 538 back to the second reactor 502 through conduit 541. The gaseous combustion products are released from the catalyst regenerator 538 through flue gas stream 540. In another embodiment, not shown, the regenerated catalyst composition additionally or alternatively is directed, optionally in a fluidized manner, from the catalyst regenerator 538 to one or more of the second reactor 502 and/or the catalyst stripper 534. In one embodiment, not shown, a portion of the catalyst composition in the reaction system is transported directly, e.g., without first passing through the catalyst stripper 534, optionally in a fluidized manner, from the second reactor 502 to the catalyst regenerator 538.

As the catalyst compositions contact the regeneration medium in catalyst regenerator 538, the temperature of the catalyst composition will increase due to the exothermic nature of the regeneration process. As a result, it is desirable to control the temperature of the catalyst composition by directing at least a portion of the catalyst composition from the catalyst regenerator 538 to a catalyst cooler 543. As shown, the catalyst composition is transported in a fluidized manner from catalyst regenerator 538 to the catalyst cooler 543 through conduit 542. The resulting cooled catalyst composition is transported, preferably in a fluidized manner, from catalyst cooler 543 back to the catalyst regenerator 538 through conduit 544. In another embodiment, not shown, the cooled catalyst composition additionally or alternatively is directed, optionally in a fluidized manner, from the catalyst cooler 543 to one or more of the second reactor 502 and/or the catalyst stripper 534.

In another embodiment, not shown, all or a portion of the first effluent 552 is added to and combined with second effluent 504 to form a combined stream, which is directed to quench tower 506. In this embodiment, the water in the first and second effluent streams 552 and 504 is removed in quench tower 506.

3. Converting a Mixed Feedstock to Light Olefins in a Single Reactor Utilizing a Mixture of Catalyst Particles

In another embodiment, the invention is to a process for producing light olefins, the process comprising the steps of: (a) providing a feedstock comprising methanol and ethanol; and (b) fluidizing a population of catalyst particles in a fluidized reactor with the feedstock under conditions effective to convert the methanol and the ethanol to light olefins and water, wherein the population of catalyst particles comprises ETE catalyst particles and molecular sieve catalyst particles. In this embodiment, the light olefins comprise ethylene and propylene, and the weight ratio of ethylene to propylene formed in step (b) optionally is greater than about 0.7, preferably greater than about 1.0, and most preferably greater than about 1.2.

One benefit of this embodiment is that it reduces the number of required units in the reaction system. Additionally, little or no modification of an existing OTO reaction system is necessary to implement this embodiment of the present invention. It is contemplated, however, that some methanol degradation undesirably may occur over the ETE catalyst particles in this aspect of the invention.

In operation, this aspect of the invention preferably would resemble a conventional MTO reaction system, as illustrated, for example, in FIG. 1. The principle difference is that the feedstock comprises a combination of methanol and ethanol and the population of catalyst particles contained in the reaction system comprises ETE catalyst particles as well as MTO catalyst particles.

Additionally, this embodiment of the present invention also allows for considering thermodynamic considerations of the MTO and ETE reaction processes. The conversion of methanol to light olefins (MTO) is slightly exothermic in nature while the conversion of ethanol to ethylene (ETE) is endothermic in nature. It has now been discovered that by directing methanol and ethanol to an OTO reaction zone in the preferred weight ratios indicated above, the net heat of reactions, ΔHₙₑₜ, for the conversion of the methanol and ethanol to light olefins can be advantageously balanced for maximum ethylene production without adding additional heat to the reaction zone. That is, heat evolved from the exothermic conversion of methanol to light olefins is utilized in the endothermic conversion of ethanol to ethylene thereby providing a commensurate increase in olefin selectivity and alcohol conversion. Additionally, the light olefins formed in the reaction zone are desirably rich in ethylene, which typically is more valuable than propylene, compared to the light olefins formed from a feedstock comprising about 100 wt. % methanol, as discussed in greater detail above.

It has been discovered that at greater than about 12.5 weight percent ethanol content (balance methanol), the heat requirements of the ETE reaction have a negative impact on the simultaneously occurring MTO reaction, and the amount of light olefins produced by the MTO reaction decreases. As a result, without adding heat to the reaction system, total prime olefin selectivity drops off at ethanol levels greater than about 12.5 weight percent ethanol. Thus, the feedstock preferably comprises about 12 or more particularly about 12.5 weight percent ethanol, the balance preferably substantially comprising methanol.

The amount of ETE catalyst particles relative to MTO catalyst particles in a reaction system according to this embodiment of the present invention may vary widely. As indicated above, in a mixed catalyst system, some methanol degradation, e.g., to mehane, may occur over the ETE catalyst particles. Preferably, the ratio of ETE catalyst particles to MTO catalyst particles in the reaction is kept low enough so as to limit degradation of methanol in the feedstock to less than about 5 weight percent, less than about 2 weight percent, or less than about 1 weight percent, based on the total amount of methanol in the feedstock. By degradation, it is meant the conversion of methanol to non-olefin compounds.

In another embodiment, the amount of ETE catalyst particles relative to MTO catalyst particles in a reaction system according to this embodiment is adapted to correspond with the preferred methanol to ethanol ratios of the feedstock, discussed above. In one embodiment, for example, the population of catalyst particles comprises from about 2 to about 22 weight percent ETE catalyst particles, more preferably from about 8 to about 16 weight percent ETE catalyst particles, based on the total weight of the population of catalyst particles—the balance preferably comprising the MTO catalyst particles. These ratios are particularly preferred because they correspond with the preferred ratios of methanol and ethanol in the feedstock, as described above.

In any of the above-described processes of the present invention, the ETE catalyst particles optionally are selected from the group consisting of: silica-alumina catalyst particles, activated alumina catalyst particles and activated clay catalyst particles. In any of the above-described processes of the present invention, the molecular sieve catalyst particles optionally comprise a molecular sieve selected from the group consisting of: SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, AEI/CHA intergrowths, metal containing forms thereof, intergrown forms thereof, and mixtures thereof.

Having now fully described the invention, it will be appreciated by those skilled in the art that the invention may be performed within a wide range of parameters within what is claimed, without departing from the spirit and scope of the present invention

We claim:

1. A process for producing light olefins, the process comprising the steps of:
(a) providing a feedstock comprising methanol and ethanol;
(b) dehydrating at least a portion of the ethanol in a first reactor to form a first effluent comprising ethylene, methanol, water and less than about 2 weight percent acetaldehyde, based on the total weight of the first effluent; and
(c) contacting the methanol in the first effluent with a molecular sieve catalyst composition in a second reactor under conditions effective to convert the methanol to additional light olefins.

2. The process of claim 1, wherein the molecular sieve catalyst composition comprises a molecular sieve selected from the group consisting of: SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, AEI/CHA intergrowths, metal containing forms thereof, intergrown forms thereof, and mixtures thereof.

3. The process of claim 1, wherein the cumulative amount of ethylene and propylene formed in steps (b) and (c) has a weight ratio of ethylene to propylene of greater than about 0.7.

4. The process of claim 3, wherein the weight ratio of ethylene to propylene is greater than about 1.0.

5. The process of claim 4, wherein the weight ratio of ethylene to propylene is greater than about 1.2.

6. The process of claim 1, wherein the methanol to ethanol weight ratio in the feedstock is from about 1 to about 100.

7. The process of claim 6, wherein the methanol to ethanol weight ratio is from about 3 to about 20.

8. The process of claim 1, wherein step (b) comprises contacting the ethanol with a dehydration catalyst under conditions effective to convert the ethanol to the ethylene and water, wherein the dehydration catalyst is selected from the group consisting of: silica-alumina, activated alumina, phosphoric acid, and activated clay.

9. The process of claim 1, wherein the process further comprises the step of:
(d) removing a weight majority of the water from the first effluent between steps (b) and (c).

10. The process of claim 1, wherein the first effluent comprises less than about 1 weight percent acetaldehyde.

11. The process of claim 10, wherein the first effluent comprises less than about 0.2 weight percent acetaldehyde.

12. The process of claim 1, wherein the first effluent comprises at least about 5 weight percent methanol.

13. The process of claim 12, wherein the first effluent comprises at least about 25 weight percent methanol.

14. The process of claim 1, wherein the first effluent comprises at least about 5 weight percent ethylene.

15. The process of claim 14, wherein the first effluent comprises at least about 10 weight percent ethylene.

16. The process of claim 1, wherein at least a portion of the methanol from the feedstock is dehydrated to dimethyl ether in the first reactor, and wherein the first effluent further comprises the dimethyl ether.

17. The process of claim 16, wherein the first effluent comprises at least about 5 weight percent dimethyl ether.

18. The process of claim 17, wherein the first effluent comprises at least about 25 weight percent dimethyl ether.

19. The process of claim 16, wherein the process further comprises the step of:
(d) contacting at least a portion of the dimethyl ether with the molecular sieve catalyst composition in the second reactor under conditions effective to convert the dimethyl ether to ethylene.

20. The process of claim 1, wherein a weight majority of the methanol from the feedstock passes through the first reactor and into the first effluent.

21. The process of claim 1, wherein the first reactor comprises an alcohol dehydration reactive distillation column.

22. The process of claim 21, wherein a weight majority of the water formed in step (b) is separated in the distillation column from a weight majority of the methanol and ethylene, collectively, formed in step (b).

23. A process for producing light olefins, the process comprising the steps of:
(a) providing a feedstock comprising methanol and ethanol;
(b) separating the feedstock into a methanol-containing stream and an ethanol-containing stream, wherein the methanol-containing stream comprises a weight majority of the methanol from the feedstock, and the ethanol-containing stream comprises a weight majority of the ethanol from the feedstock;
(c) contacting the ethanol in the ethanol-containing stream with a dehydration catalyst in a first reactor under conditions effective to convert the ethanol to water and light olefins, wherein the light olefins are yielded from the first reactor in a first effluent;
(d) contacting the methanol in the methanol-containing stream with a molecular sieve catalyst composition in a second reactor under conditions effective to convert the methanol to light olefins and water, which are yielded from the second reactor in a second effluent; and
(e) combining at least a portion of the first effluent with at least a portion of the second effluent to form a combined product stream.

24. The process of claim 23, wherein the molecular sieve catalyst composition comprises a molecular sieve selected from the group consisting of: SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, AEI/CHA intergrowths, metal containing forms thereof, intergrown forms thereof, and mixtures thereof.

25. The process of claim 23, wherein the cumulative amount of ethylene and propylene formed in steps (c) and (d) has a weight ratio of ethylene to propylene of greater than about 0.7.

26. The process of claim 25, wherein the weight ratio of ethylene to propylene is greater than about 1.0.

27. The process of claim 26, wherein the weight ratio of ethylene to propylene is greater than about 1.2.

28. The process of claim 23, wherein the methanol to ethanol weight ratio in the feedstock is from about 1 to about 100.

29. The process of claim 28, wherein the methanol to ethanol weight ratio is from about 3 to about 20.

30. The process of claim 23, wherein the dehydration catalyst is selected from the group consisting of: silica-alumina, activated alumina, phosphoric acid, and activated clay.

31. The process of claim 23, wherein the first effluent comprises less than 1 weight percent acetaldehyde.

32. The process of claim 31, wherein the first effluent comprises less than 0.2 weight percent acetaldehyde.

33. The process of claim 23, wherein the first reactor comprises an alcohol dehydration reactive distillation column.

34. The process of claim 33, wherein the alcohol dehydration reactive distillation column separates a weight majority of the light olefins formed in step (c) from a weight majority of the water formed in step (c), wherein the first effluent comprises the weight majority of the light olefins.

35. The process of claim 23, wherein the first reactor comprises a fixed bed dehydration reactor.

36. The process of claim 23, wherein the first effluent further comprise the water formed in step (c).

37. The process of claim 23, wherein the feedstock further comprises one or more C3+ alcohols, a weight majority of which are separated in step (b) into the ethanol-containing stream, and which C3+ alcohols are also dehydrated to light olefins and water in the first reactor.

38. The process of claim 37, wherein the feedstock comprises more than 1 weight percent C3+ alcohols, based on the weight of the feedstock.

39. The process of claim 23, wherein the feedstock further comprises greater than about 1 weight percent water, based on the total weight of the feedstock.

40. The process of claim 39, wherein the feedstock further comprises greater than about 10 weight percent water, based on the total weight of the feedstock.

41. A process for producing light olefins, the process comprising the steps of:
(a) providing a feedstock comprising methanol and ethanol; and
(b) contacting a population of catalyst particles in a fluidized reactor with the feedstock under conditions effective to convert the methanol and the ethanol to light olefins and water, wherein the population of catalyst particles comprises ETE catalyst particles and molecular sieve catalyst particles.

42. The process of claim 41, wherein the population of catalyst particles comprises from about 2 to about 22 weight percent ETE catalyst particles, based on the total weight of the population of catalyst particles.

43. The process of claim 42, wherein the population of catalyst particles comprises from about 8 to about 16 weight percent ETE catalyst particles, based on the total weight of the population of catalyst particles.

44. The process of claim 41, wherein the ETE catalyst particles are selected from the group consisting of: silica-alumina catalyst particles, activated alumina catalyst particles, solid phosphoric acid, and activated clay catalyst particles.

45. The process of claim 42, wherein the molecular sieve catalyst particles comprise a molecular sieve selected from the group consisting of: SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, AEI/CHA intergrowths, metal containing forms thereof, intergrown forms thereof, and mixtures thereof.

46. The process of claim 41, wherein the light olefins comprise ethylene and propylene, and the weight ratio of ethylene to propylene formed in step (b) is greater than about 0.7.

47. The process of claim 46, wherein the weight ratio of ethylene to propylene is greater than about 1.0.

48. The process of claim 47, wherein the weight ratio of ethylene to propylene is greater than about 1.2.

49. The process of claim 41, wherein the methanol to ethanol weight ratio in the feedstock is from about 1 to about 100.

50. The process of claim 49, wherein the methanol to ethanol weight ratio in the feedstock is from about 3 to about 20.

51. The process of claim 41, wherein the light olefins and water formed in step (b) are yielded from the fluidized reactor in an effluent stream comprising less than 1 weight percent acetaldehyde, based on the total weight of the effluent stream.

52. The process of claim 51, wherein the effluent stream comprises less than 0.2 weight percent acetaldehyde, based on the total weight of the effluent stream.

53. The process of claim 41, wherein the feedstock further comprises greater than about 1 weight percent water, based on the total weight of the feedstock.

54. The process of claim 53, wherein the feedstock further comprises greater than about 10 weight percent water, based on the total weight of the feedstock.
